(19) United States
(12) Patent Application Publication
(10) Pub. No.: US 2007/0166810 A1
(43) Pub. Date: Jul. 19, 2007

(54) METHODS AND COMPOSITIONS FOR DETECTING ENZYMATIC ACTIVITY

(75) Inventors: Mark N. Bobrow, Lexington, MA (US); Karl Edwin Adler JR., Newburyport, MA (US); Mack J. Schermer, Belmont, MA (US)

Correspondence Address: FISH & RICHARDSON PC, P.O. BOX 1022, MINNEAPOLIS, MN, 55440-1022, US

(73) Assignee: PERKINELMER LAS, INC., Boston, MA (US)

(21) Appl. No.: 11/615,733

(22) Filed: Dec. 22, 2006

Related U.S. Application Data
(60) Provisional application No. 60/753,492, filed on Dec. 23, 2005.
(60) Provisional application No. 60/753,583, filed on Dec. 23, 2005.

(51) Int. Cl. C12N 9/00

(52) U.S. Cl. 435/183

(57) ABSTRACT

This invention relates to methods and compositions useful in detecting enzymatic activity. Also featured are methods for diagnosing enzyme deficiencies such as metabolic disorders.

### CROSS-REFERENCE TO RELATED APPLICATIONS

This application claims priority to U.S. application Ser. No. 60/753,492, filed on Dec. 23, 2005, and Ser. No. 60/753,583, filed on Dec. 23, 2005, the contents of which are hereby incorporated by reference.

### TECHNICAL FIELD

This invention relates, inter alia, to methods and compositions useful in detecting enzymatic activity. Also featured are methods and compositions for diagnosing enzyme deficiencies such as metabolic disorders.

### SUMMARY

The technology described herein relates, inter alia, to methods and compositions for detecting the presence or amount of an enzymatic activity in a sample using supports, e.g., particles. The methods and compositions can be used for assessing the presence or amount of a variety of enzymatic activities, and can be used in multiplexed formats, if desired. Information about enzymatic activity can be used, for example, to assess the presence or amount of an enzyme.

In one aspect, the disclosure features a method for evaluating multiple enzymatic activities in a sample. The method can include: providing a mixture of encoded particles comprising particles having an attached enzymatic substrate, the enzymatic substrate being identifiable by a particle code, wherein the mixture includes particles for a plurality of different enzymatic substrates; contacting the mixture of particles with a sample; and detecting modification of at least one of the enzymatic substrates attached to the particles for at least some of the encoded particles.

In some embodiments, modification of at least one of the enzymatic substrates attached to the particles can produce a particle-bound product portion from the enzymatic substrate.

In some embodiments, detecting can include contacting the mixture of particles to a mixture comprising one or more enzymatic substrate-binding partners, wherein the one or more enzymatic substrate-binding partners are detectably-labeled and detect support-bound product portions from enzymatic substrates.

In some embodiments, detecting can include contacting the mixture of particles to a mixture comprising one or more enzymatic substrate-binding partners, wherein the one or more enzymatic substrate-binding partners are detectably-labeled and detect unmodified enzymatic substrates. In some embodiments, the detecting can further include evaluating interaction of enzymatic substrate binding partners with the particles and the particle codes using a flow cytometer.

In some embodiments, detecting can include determining the amount of modification of at least one of the particle-bound enzymatic substrates.

In some embodiments, detection can include detecting the presence or amount of at least one unmodified particle-bound enzymatic substrate.

In some embodiments, the particles can be magnetic.

In some embodiments, at least one enzymatic substrate can contain a linker moiety.

In some embodiments, the sample can be obtained from cultured cells. In some embodiments, the sample can be obtained from a mammal such as a human. In some embodiments the human can be a newborn baby. In some embodiments, the human can be one having, or suspected of having, a metabolic disorder. The metabolic disorder can acid lipase disease, amyloidosis, Barth Syndrome, biotinidase deficiency, carnitine palitoyl transferase deficiency type II (CPT-II), central pontine myelinolysis, Farber's disease, Fabry's disease, Pompe's disease, glucose-6-phosphate dehydrogenase deficiency, gangliosidoses, trimethylaminuria, Lesch-Nyhan Syndrome, methylmalonic aciduria (MMA), mucopolysaccharidosis, mucolipodosis, multiple CoA carboxylase deficiency (MCCD), nonketotic hyperglycinemia (NKH), propionic acidemia (PROP), type I glycogen storage disease, hyperoxaluria, oxalosis, 3-methylcrotonyl-COA carboxylase deficiency (3MCC), 3-OH 3-CH3 glutaric aciduria (HMG), arginosuccinic acidemia (ASA), beta-ketothiolase deficiency (BKT), 21-hydroxylase deficiency, CBS deficiency, maple syrup urine disease (MSUD), phenylketonuria (PKU), or very long-chain acyl-CoA dehydrogenase deficiency (VLCADD).

In some embodiments, the sample can be, or contain, a biological fluid such as blood, plasma, or serum.

In some embodiments, the particle-bound enzymatic substrate comprises a detectable tag.

In some embodiments, the mixture of particles can be contacted with more than one sample in parallel.

In another aspect, the disclosure provides a method for detecting hydrolase activity, which can include the steps of: providing a hydrolase substrate bound to a support; contacting the support-bound hydrolase substrate with a sample; and detecting modification of the support-bound hydrolase substrate.

In some embodiments, the detecting can include determining the amount of modification of the support-bound hydrolase substrate.

In some embodiments, the detecting can include detecting the presence or amount of unmodified support-bound hydrolase substrate.

In some embodiments, the support is a magnetic particle or an encoded particle. In some embodiments, the support can be a magnetic and encoded particle.

In some embodiments, the hydrolase substrate can contain a linker moiety.

In some embodiments, the hydrolase can be a biotinidase. In some embodiments, for example where the hydrolase is a biotinidase, the substrate can be, or contain, biotin or biocytin.

In some embodiments, the sample can be obtained from cultured cells. In some embodiments, the sample can be obtained from a mammal such as a human. In some embodiments the human can be a newborn baby. In some embodiments, the human can be one having, or suspected of having, a metabolic disorder. The metabolic disorder can acid lipase disease, amyloidosis, Barth Syndrome, biotinidase deficiency, carnitine palitoyl transferase deficiency type II (CPT-II), central pontine myelinolysis, Farber's disease, Fabry's disease, Pompe's disease, glucose-6-phosphate dehydrogenase deficiency, gangliosidoses, trimethylaminuria, Lesch-Nyhan Syndrome, methylmalonic aciduria (MMA), mucopolysaccharidosis, mucolipodosis, multiple CoA carboxylase deficiency (MCCD), nonketotic hyperglycinemia (NKH), propionic acidemia (PROP), type I glycogen storage disease, hyperoxaluria, oxalosis, 3-methylcrotonyl-COA carboxylase deficiency (3MCC), 3-OH 3-CH3 glutaric aciduria (HMG), arginosuccinic acidemia (ASA), beta-ketothiolase deficiency (BKT), 21-hydroxylase deficiency, CBS deficiency, maple syrup urine disease (MSUD), phenylketonuria (PKU), or very long-chain acyl-CoA dehydrogenase deficiency (VLCADD).

In some embodiments, the sample can be, or contain, a biological fluid such as blood, plasma, or serum.

In some embodiments, the support-bound hydrolase substrate can contain a detectable tag. In some embodiments, detecting can include contacting the support-bound hydrolase substrate with a detectably-labeled hydrolase substrate-binding partner. In some embodiments, the hydrolase substrate-binding partner can be labeled with a fluorescent moiety. In some embodiments, the hydrolase substrate-binding partner can contain a member of a specific binding-pair. The specific binding pair can be biotin and streptavidin.

In some embodiments, a support-bound hydrolase substrate can be contacted with more than one sample in parallel.

In another aspect, the disclosure features a method for detecting biotinidase activity. The method can include the steps of: providing a biotinidase substrate bound to a support; contacting the support-bound biotinidase substrate with a sample; and detecting modification of the support-bound biotinidase substrate. For example, the support is a magnetic particle or an encoded particle, e.g., a magnetic encoded particle.

In some embodiments, the detecting can include determining the amount of modification of the support-bound biotinidase substrate.

In some embodiments, the detecting can include detecting the presence or amount of unmodified support-bound biotinidase substrate.

In some embodiments, the biotinidase substrate can contain a linker moiety. In some embodiments, the substrate can be, or contain, biotin or biocytin.

In some embodiments, the sample can be obtained from cultured cells. In some embodiments, the sample can be obtained from a mammal such as a human. In some embodiments, the human can be a newborn baby. In some embodiments, the human can be one having, or suspected of having, a biotinidase deficiency. In some embodiments, the sample can be, or contain, a biological fluid such as blood, plasma, or serum.

In some embodiments, the support-bound biotinidase substrate can contain a detectable tag. In some embodiments, detecting can include contacting the support-bound biotinidase substrate with a detectably-labeled biotinidase substrate-binding partner. In some embodiments, the biotinidase substrate-binding partner can be labeled with a fluorescent moiety. In some embodiments, the biotinidase substrate-binding partner can contain, or be, avidin or streptavidin.

In some embodiments, a support-bound biotinidase substrate can be contacted with more than one sample in parallel.

In another aspect, the disclosure features a method for detecting biotinidase activity, which can include the steps of: providing biotin bound to a support; contacting the support-bound biotin with a sample; contacting the support-bound biotin with a detectably-labeled biotin-binding moiety; and detecting binding of the biotin-binding moiety to the support-bound biotin.

In some embodiments, detecting can include determining the amount of biotin-binding moiety bound to the support-bound biotin.

In some embodiments, the support can be magnetic, such as a magnetic particle. In some embodiments, the support can be encoded, such as an encoded particle. In some embodiments, the support can be magnetic and encoded such as a magnetic and encoded particle.

In some embodiments, biotin can contain a linker moiety.

In some embodiments, the sample can be obtained from cultured cells. In some embodiments, the sample can be obtained from a mammal such as a human. In some embodiments the human can be a newborn baby. In some embodiments, the human can be one having, or suspected of having, a biotinidase deficiency.

In some embodiments, the sample can be, or contain, a biological fluid such as blood, plasma, or serum.

In some embodiments, the biotin-binding partner can be labeled with a fluorescent moiety. In some embodiments, the biotin-binding partner can be, or contain, streptavidin or avidin.

In some embodiments, the support-bound biotin can be contacted with more than one sample in parallel.

In yet another aspect, the disclosure provides a method for detecting hydrolase activity. The method can include the steps of: providing particles to which a hydrolase substrate is attached; contacting the particles with a sample; contacting the particles with a fluorescently labeled intact substrate-binding partner; and detecting binding of the intact substrate-binding partner to the support by evaluating fluorescence associated with one or more of the particles.

In some embodiments, the particles can be magnetic or encoded. In some embodiments, the particles can be both magnetic and encoded.

In some embodiments, the hydrolase substrate can contain a linker moiety.

In some embodiments, the sample can be obtained from cultured cells. In some embodiments, the sample can be obtained from a mammal such as a human. In some embodiments the human can be a newborn baby. In some embodiments, the human can be one having, or suspected of having, a metabolic disorder. The metabolic disorder can acid lipase disease, amyloidosis, Barth Syndrome, biotinidase deficiency, carnitine palitoyl transferase deficiency type II (CPT-II), central pontine myelinolysis, Farber's disease, Fabry's disease, Pompe's disease, glucose-6-phosphate dehydrogenase deficiency, gangliosidoses, trimethylaminuria, Lesch-Nyhan Syndrome, methylmalonic aciduria (MMA), mucopolysaccharidosis, mucolipodosis, multiple CoA carboxylase deficiency (MCCD), nonketotic hyperglycinemia (NKH), propionic acidemia (PROP), type I glycogen storage disease, hyperoxaluria, oxalosis, 3-methylcrotonyl-COA carboxylase deficiency (3MCC), 3-OH 3-CH3 glutaric aciduria (HMG), arginosuccinic acidemia (ASA), beta-ketothiolase deficiency (BKT), 21-hydroxylase deficiency, CBS deficiency, maple syrup urine disease (MSUD), phenylketonuria (PKU), or very long-chain acyl-CoA dehydrogenase deficiency (VLCADD).

In some embodiments, the sample can be, or contain, a biological fluid such as blood, plasma, or serum.

In some embodiments, the support-bound hydrolase substrate can contain a detectable tag. In some embodiments, detecting can include contacting the support-bound hydrolase substrate with a detectably-labeled hydrolase substrate-binding partner. In some embodiments, the hydrolase substrate-binding partner can be labeled with a fluorescent moiety. In some embodiments, the hydrolase substrate-binding partner can contain a member of a specific binding-pair. The specific binding pair can be biotin and streptavidin.

In some embodiments, a support-bound hydrolase substrate can be contacted with more than one sample in parallel.

In another aspect, the disclosure features a method for detecting biotinidase activity, which can include the steps of: providing particles to which biotin is attached; contacting the particles with a sample; contacting the particles with a fluorescently labeled biotin-binding partner; and detecting binding of the biotin-binding partner to the support by evaluating fluorescence associated with one or more of the particles.

In some embodiments, detecting can include determining the amount of biotin-binding moiety bound to the support-bound biotin.

In some embodiments, the support can be magnetic, such as a magnetic particle. In some embodiments, the support can be encoded, such as an encoded particle. In some embodiments, the support can be magnetic and encoded such as a magnetic and encoded particle.

In some embodiments, biotin can contain a linker moiety.

For example, the sample can be obtained from cultured cells or a biological fluid such as blood, plasma, or serum. In some embodiments, the sample can be obtained from a mammal such as a human. In some embodiments the human can be a newborn baby. In some embodiments, the human can be one having, or suspected of having, a biotinidase deficiency.

In some embodiments, the biotin-binding partner can be labeled with a fluorescent moiety. In some embodiments, the biotin-binding partner can be, or contain, streptavidin or avidin.

In some embodiments, the particle-bound biotin can be contacted with more than one sample in parallel.

In another aspect, the disclosure features a kit for detecting biotinidase activity. The kit can include: (i) a support-bound biotinidase substrate; and, optionally, (ii) instructions for detecting biotinidase activity.

In some embodiments, the support can be magnetic or encoded. In some embodiments, the support can be both magnetic and encoded. In some embodiments, the support can be a particle.

In some embodiments, the biotinidase substrate can contain a linker moiety. In some embodiments, the biotinidase substrate can be, or contain, biotin or biocytin.

In another aspect, the disclosure provides a diagnostic method for evaluating biotinidase deficiency in a subject, which method includes the steps of: providing a biotinidase substrate bound to a support; providing a sample from a subject; contacting the support-bound biotinidase substrate with the sample; and detecting whether modification of the support-bound biotinidase substrate has occurred.

In some embodiments, the method can further include the step of: providing a result indicating whether the subject has a biotinidase deficiency, wherein the result is a function of the extent of reduction in modification of the support-bound biotinidase substrate, relative to a reference of normal control.

In some embodiments, the subject is a mammal such as a human. In some embodiments, the subject can be a newborn, for example, a newborn human. In some embodiments, the subject can be one having, or suspected of having, a genetic predisposition to a biotinidase deficiency.

In some embodiments, the method can further include the step of after evaluating the subject for a biotinidase deficiency, administering to the subject a therapeutically effective amount of biotin.

In yet another aspect, the disclosure features a method of detecting an enzymatic activity that catalyzes a reaction. The method can include the steps of: contacting an enzymatic substrate with a sample and a mixture comprising particles, wherein at least a portion of the particles preferentially bind to a reaction component; and detecting the support-bound reaction component. For example, at least of the particles can be encoded or magnetic. In some embodiments, at least some of the particles can be both magnetic and encoded.

In some embodiments, the reaction component can be the enzymatic substrate. In some embodiments, the reaction component can be a reaction product produced from the enzyme-catalyzed reaction.

In some embodiments, the enzymatic substrate can be contacted with the sample and then contacted with the mixture. In some embodiments, the enzymatic substrate and mixture can be contacted to more than one sample in parallel.

In some embodiments, the enzymatic substrate can be, or contain, fibrinogen. In some embodiments, the enzymatic substrate can be, or contain, biotin or biocytin. In some embodiments, the enzymatic substrate can contain a linker moiety and/or a detectable tag.

In some embodiments, the sample can be obtained from cultured cells. In some embodiments, the sample can be obtained from a mammal such as a human. In some embodiments the human can be a newborn baby. In some embodiments, the human can be one having, or suspected of having, a metabolic disorder. The metabolic disorder can acid lipase disease, amyloidosis, Barth Syndrome, biotinidase deficiency, carnitine palitoyl transferase deficiency type II (CPT-II), central pontine myelinolysis, Farber's disease, Fabry's disease, Pompe's disease, glucose-6-phosphate dehydrogenase deficiency, gangliosidoses, trimethylaminuria, Lesch-Nyhan Syndrome, methylmalonic aciduria (MMA), mucopolysaccharidosis, mucolipodosis, multiple CoA carboxylase deficiency (MCCD), nonketotic hyperglycinemia (NKH), propionic acidemia (PROP), type I glycogen storage disease, hyperoxaluria, oxalosis, 3-methylcrotonyl-COA carboxylase deficiency (3MCC), 3-OH 3-CH3 glutaric aciduria (HMG), arginosuccinic acidemia (ASA), beta-ketothiolase deficiency (BKT), 21-hydroxylase deficiency, CBS deficiency, maple syrup urine disease (MSUD), phenylketonuria (PKU), or very long-chain acyl-CoA dehydrogenase deficiency (VLCADD).

In some embodiments, the sample can be, or contain, a biological fluid such as blood, plasma, or serum.

In yet another aspect, the disclosure features a method for detecting enzymatic activity. The method can include the steps of: contacting a support-bound enzymatic substrate with a sample; and detecting whether modification of the support-bound enzymatic substrate has occurred.

In some embodiments, the detecting can include determining the amount of modification of the support-bound enzymatic substrate. In some embodiments, the detecting can include detecting the presence or amount of unmodified support-bound biotinidase substrate.

In some embodiments, the support can be magnetic (such as a magnetic particle) or encoded (such as an encoded particle). In some embodiments, the support can be both magnetic and encoded.

In some embodiments, the sample can be obtained from cultured cells. In some embodiments, the sample can be obtained from a mammal such as a human. In some embodiments the human can be a newborn baby. In some embodiments, the human can be one having, or suspected of having, a metabolic disorder. The metabolic disorder can acid lipase disease, amyloidosis, Barth Syndrome, biotinidase deficiency, carnitine palitoyl transferase deficiency type II (CPT-II), central pontine myelinolysis, Farber's disease, Fabry's disease, Pompe's disease, glucose-6-phosphate dehydrogenase deficiency, gangliosidoses, trimethylaminuria, Lesch-Nyhan Syndrome, methylmalonic aciduria (MMA), mucopolysaccharidosis, mucolipodosis, multiple CoA carboxylase deficiency (MCCD), nonketotic hyperglycinemia (NKH), propionic acidemia (PROP), type I glycogen storage disease, hyperoxaluria, oxalosis, 3-methylcrotonyl-COA carboxylase deficiency (3MCC), 3-OH 3-CH3 glutaric aciduria (HMG), arginosuccinic acidemia (ASA), beta-ketothiolase deficiency (BKT), 21-hydroxylase deficiency, CBS deficiency, maple syrup urine disease (MSUD), phenylketonuria (PKU), or very long-chain acyl-CoA dehydrogenase deficiency (VLCADD).

In some embodiments, the sample can be, or contain, a biological fluid such as blood, plasma, or serum.

In some embodiments, the support-bound enzymatic substrate can contain a detectable tag and/or a linker moiety.

In some embodiments, detecting can include contacting the support-bound enzymatic substrate with a detectably-labeled enzymatic substrate-binding partner. In some embodiments, detecting can include contacting the support-bound enzymatic substrate with a detectably-labeled support-bound product-binding partner.

In some embodiments, the support-bound enzymatic substrate is contacted with more than one sample in parallel. In some embodiments, more than one different support-bound enzymatic substrates can be provided. In some embodiments, for example where more than one support-bound enzymatic substrate is provided, the support-bound enzymatic substrates can be contacted with a sample in parallel.

In another aspect, the disclosure features a diagnostic method for evaluating a metabolic disorder resulting from an enzyme deficiency in a subject, which method can include the steps of: providing an enzymatic substrate bound to a support; providing a sample from a subject; contacting the support-bound enzymatic substrate with the sample; and detecting whether modification of the support-bound enzymatic substrate has occurred.

In some embodiments, the method can also include the step of providing a result indicating whether the subject has a metabolic disorder, wherein the result is a function of the extent of reduction in modification of the support-bound enzymatic substrate, relative to a reference of normal control.

In some embodiments, the subject can be a human. In some embodiments the subject can be a newborn, such as a newborn human child. The subject can be one having, or suspected of having, a genetic predisposition to a metabolic disorder. The metabolic disorder can be: acid lipase disease, amyloidosis, Barth Syndrome, biotinidase deficiency, carnitine palitoyl transferase deficiency type II (CPT-II), central pontine myelinolysis, Farber's disease, Fabry's disease, Pompe's disease, glucose-6-phosphate dehydrogenase deficiency, gangliosidoses, trimethylaminuria, Lesch-Nyhan Syndrome, methylmalonic aciduria (MMA), mucopolysaccharidosis, mucolipodosis, multiple CoA carboxylase deficiency (MCCD), nonketotic hyperglycinemia (NKH), propionic acidemia (PROP), type I glycogen storage disease, hyperoxaluria, oxalosis, 3-methylcrotonyl-COA carboxylase deficiency (3MCC), 3-OH 3-CH3 glutaric aciduria (HMG), arginosuccinic acidemia (ASA), beta-ketothiolase deficiency (BKT), 21-hydroxylase deficiency, CBS deficiency, maple syrup urine disease (MSUD), phenylketonuria (PKU), or very long-chain acyl-CoA dehydrogenase deficiency (VLCADD).

All publications, patent applications, patents, and other references mentioned herein are incorporated by references in their entirety. The materials, methods, and examples disclosed herein are illustrative only and not intended to be limiting.

Other features and advantages of the invention will be apparent from the following description, from the drawings, and from the claims.

### BRIEF DESCRIPTION OF THE DRAWINGS

FIGS. 1A-1E show schematics of enzyme assay components useful in an exemplary method described herein. FIG. 1A shows a particle displaying intact substrate. FIG. 1B shows a particle displaying intact substrate in the presence of an enzyme and two product, one that is free and another that is bound to the particle. FIG. 1C shows a particle displaying bound product portions. FIG. 1D shows a particle displaying bound product portions as well as complexes of intact substrate bound to a detectable tag via an intact-substrate-binding partner. FIG. 1E shows a particle displaying complexes of intact substrate bound to detectable tag via intact-substrate-binding partner.

FIG. 2 shows experimental results of an exemplary biotinidase assay performed according to a method described herein.

FIG. 3 depicts an exemplary layout of reagents in microplate wells in a pattern according to one aspect of the present invention.

FIGS. 4A-41 are schematic drawings showing a progression of steps of manipulating magnetic assay beads through several assay steps by transferring them from one reagent-filled vessel to another in a pattern.

### DETAILED DESCRIPTION

The methods described herein involve assaying enzymatic activity, for example, by detecting a reaction component (e.g., a substrate or product) attached to a support. The methods can be used, for example, to assess the level of enzymatic activity in the sample.

The substrate used in the enzymatic reaction can be referred to as an “intact substrate,” merely to distinguish it from enzymatic reaction products that are modified versions of the substrate. Typically intact substrates are attached to a support, such as a particle (see below), dipstick, filter, or sample receptacle. The sample is contacted to the support and the support is evaluated to determine if the intact substrate is modified. For example, under suitable conditions, a cognate enzyme with a cleavage activity will cleave the intact substrate. In one scenario, cleavage of an intact substrate can produce at least two products, one being a “support-bound product,” which remains on the support, and the other being a “free product,” which is released from the support, either or both of which can be detectable. Thus, a typical reaction mixture can contain one or more of: a support-bound intact substrate, a support-bound product and a free product. Any of these components can be detected to monitor the reaction.

In addition, modification of an enzymatic substrate in an enzymatic reaction can occur in the solution phase followed by the capture of one or more of reaction products onto a solid support. Reaction products can include, e.g., an unmodified enzymatic substrate or an enzymatically-derived product thereof. An example of this type of reaction involving fibrinogen is described in U.S. Pat. No. 5,583,001, which is incorporated herein by reference in its entirety.

In many embodiments, the reaction is assessed by evaluating the material bound to the support, e.g., to detect intact substrate that is not modified by enzyme, or its absence, or to detect a support-bound product. It is also possible to evaluate the released material rather than the support, e.g., to detect free product.

In another scenario, where an enzyme, for example, cleaves an intact substrate at or near its conjugation site with the support, the support-bound product is not necessarily produced. In this case, the reaction can be detected by the absence of the intact substrate or by detecting the free product (e.g., the released protein) in solution.

For example, a reduced amount of intact substrate or increased amount of either or both product portions, as compared to a negative control (such as buffer lacking enzyme) can be indicative of enzymatic activity in a sample. In some embodiments, modification of an intact substrate by an enzymatic activity produces no free product (e.g., no cleavage of an intact substrate), but does produce a support-bound product. Examples of such types of modifications can include, but are not limited to, phosphorylation, acetylation, sumolation, ubiqutination, or enantiomeric conversion. Such modifications can be evaluated by detecting the support-bound product on the support.

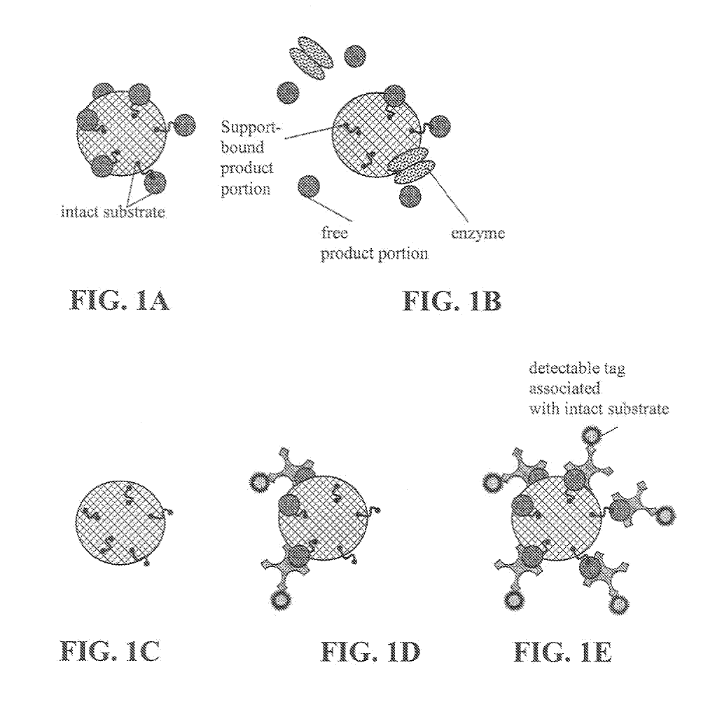

In one example, biotinidase activity in a biological sample is evaluated using an intact biotinidase substrate. See also Example 1 below. FIG. 1 illustrates various states of particles pertinent to a typical enzyme assay performed using technology described herein, including the biotinidase assay described in Example 1. FIG. 1A shows a support (e.g., a particle) displaying intact substrate. In a typical assay, a sample is contacted with the support displaying intact substrate, as is shown in FIG. 1B. The sample can be, for instance, a sample of unknown composition, one that contains or suspecting of containing an enzyme, a positive control, or a negative control. In this case, the enzymatic activity has modified the intact substrate to release a free product portion, leaving behind a support-bound product portion. Particles at this stage are typically contacted with a detectable intact substrate-binding-partner. FIGS. 1C, 1D and 1E show particles after contacting with intact substrate-binding-partner, and correspond to various outcomes of an enzyme assay. FIG. 1C shows a particle displaying support-bound product portions; no intact substrate-binding partner can bind in the absence of intact substrate, thus no detectable signal will be present. This outcome indicates the presence of an enzymatic activity at high levels that cleaved essentially all intact substrate. FIG. 1D shows a particle displaying support-bound product portions, and complexes of intact substrate bound to a detectable tag via an intact-substrate-binding partner. A detectable signal corresponding to the intact substrate is detected. This signal indicates presence of an enzyme at a moderate concentration. FIG. 1E shows a particle having complexes of intact substrate bound to detectable tag via intact-substrate-binding partner. This outcome indicates presence of an enzyme at a low concentration, or indicates the absence of enzyme activity.

Thus, some of the methods described herein involve contacting a sample with a support-bound intact substrate under conditions that allow the enzyme, if present, to modify the substrate. The sample is then evaluated by detecting support-bound intact substrate. The level of enzyme activity can be determined as a function of the amount of support-bound intact substrate. In the method illustrated in FIG. 1, the amount of support-bound intact substrate negatively correlates with the level of enzyme activity contained in the sample. The amount of support-bound intact substrate present in a reaction mixture is typically determined by comparison to controls. Examples of controls include a positive control containing a known amount of enzyme or enzyme activity and a negative control that includes the same components as the positive control except the enzyme or any buffer or composition similar to a test sample, but lacking the enzyme.

Alternatively to, or in combination with, detection of the support-bound intact substrate, the method can include detecting the presence or amount of substrate-bound-product, free product, or both. The amount of either substrate-bound product portion or free product portion typically directly correlates with the level or amount of enzymatic activity in a sample.

Activity of a variety of enzymes can be evaluated using the methods described herein. The assay is designed by selecting a substrate of the enzyme and attaching it to a support. In some embodiments, the substrate attached to the support is less than 1000, 800, or 500 daltons. If an enzyme catalyzes a reaction using two or more substrates, at least one of them and generally only one of the substrates is attached to the support. The other substrates can be added in solution along with any co-factors or other reaction compon ents.

Examples of enzymes that can be evaluated include hydrolases, lyases, oxidoreductases, transferases, isomerases and ligases. Examples of hydrolases include asparaginase, citrullinase, adenosine deaminase, creatinase, lysosomal glycoside hydrolases and the like. A specific example of a hydrolase is biotinidase. Hydrolases also include, e.g., proteases such as cysteine proteases, serine proteases, threonine proteases, aspartic acid proteases (e.g., caspases), metalloproteases, and glutamic acid proteases. Hydrolases can also include nucleases such as DNA or RNA nucleases. Other examples of enzymes that can be evaluated include dehydrogenases, oxidases, phosphatases, esterases, glycosidases, isomerases, lyases, and nucleic acid polymerases, for example, DNA polymerase I, Taq polymerase, or reverse transcriptase. The enzyme can be one whose substrate is not a polymer such as an enzyme involved in steroid biosynthesis (e.g., 17-alpha-hydroxylase/17,20 lyase, which can convert pregnenolone to its 17-alpha hydroxylated form.

A substrate for an enzyme can be any molecule that is modified by that enzyme. Typically the substrate is recognized with high affinity and specificity. For example, the substrate is the cognate substrate of the enzyme, e.g., in vivo. However, artificial substrates and mimetics of the cognate substrate can also be used. Examples of substrates include the following: biotin and biotin-based compounds for biotinidase assays, angiotensins for angiotensin converting enzyme (ACE), peptides for proteases (e.g., a thrombin-cleavable peptide for thrombin, and so for the for each of the proteases regulating coagulation). Exemplary substrates include polypeptides (e.g., short peptide sequences, large macromolecules, or complexes of one or more polypeptides), nucleic acids (e.g., DNA, RNA, or modified DNA or RNA), and small molecule compounds (e.g., steroids, vitamins). In some embodiments, the substrate attached to the support contains a heterologous moiety, such as a linker. The heterologous moiety can be one that does not affect interaction between the substrate and the enzyme.

More specifically, for biotinidase, exemplary substrates include biotin or biocytin. These substrates can also include a heterologous linker moiety, if desired. In Example 1, the attached substrate is biotin with a lysine linker that attaches it to a support.

Generally linker moieties can be used to attach any intact substrate to a support. The linker portion, or intact substrate, can contain a reactive group to facilitate chemical linkage to a support (see below). Alternatively or additionally, the linker portion, or intact substrate, can contain a moiety recognized by a binding partner that is attached to, or is attachable to, a support.

A substrate useful in a method described herein can be bound to a support. As used herein, the term “support” means a solid or semi-solid material to which an intact substrate can be bound (e.g., attached, immobilized, entrapped, captured or coated), or which can be functionalized to accept an intact substrate. The intact substrate is generally attached such that it is accessible to enzymes in solution. A support can be composed of a natural or synthetic material, an organic or inorganic material, such as a polymer, resin, metal or glass, and combinations thereof. Many suitable supports are known in the art and illustratively include particles, such as Luminex®-type encoded particles, magnetic particles, and glass particles.

A support useful in a method described herein can have a variety of physical formats, which can include for example, a membrane, column, a hollow, solid, semi-solid, pore or cavity containing particle such as a bead, a gel, a fiber, including a fiber optic material, a sheet, a matrix and sample receptacle. Examples of sample receptacles include sample wells, tubes, capillaries, vials and any other vessel, groove or indentation capable of holding a sample, including those containing membranes, filters, matrices and the like.

A sample receptacle also can be contained on a multi-sample platform, such as a microplate, slide, microfluidics device, array substrate, mass spectrometry sample plate, and the like. Exemplary particles that can be used can have a variety of sizes and physical properties. Particles can be selected to have a variety of properties useful for particular experimental formats. For example, particles can be selected that remain suspended in a solution of desired viscosity or to readily precipitate in a solution of desired viscosity. Particles can be selected for ease of separation from sample constituents, for example, by including purification tags for separation with a suitable tag-binding material, paramagnetic properties for magnetic separation, and the like.

In some embodiments, encoded particles are used. Each particle includes a unique code (such as a bar code, luminescence code, fluorescence code and the like). Encoding can be used to provide particles for evaluating different enzymatic activities in a single sample. Such methods can include contacting a sample to a mixture of encoded particles, and then contacting the encoded particles to detectably labeled tags for evaluating the enzymatic reacting. For example, the detectable tags can be used to detect the presence or amount of modified substrates (e.g., support-bound products, or free products). The code is embedded (for example, within the interior of the particle) or otherwise attached to the particle in a manner that is stable through hybridization and analysis. The code can be provided by any detectable means, such as by holographic encoding, by a fluorescence property, color, shape, size, light emission, quantum dot emission and the like to identify particle and thus the capture probes immobilized thereto. For example, the particles may be encoded using optical, chemical, physical, or electronic tags. Examples of such coding technologies are optical bar codes fluorescent dyes, or other means.

Different encoded particles can be used to evaluated a number of different enzymatic activities in parallel, so long as the encoding can be used to identify the intact substrate on a particular particle, and hence the activity being evaluated. A sample can be contacted with a plurality of such coded particles. When the particles are evaluated, e.g., using a fluorescent scanner, the particle code is read as is the fluorescence associated with the particle from any probe used to evaluate modification of the intact substrate associated with the particles.

One exemplary platform utilizes mixtures of fluorescent dyes impregnated into polymer particles as the means to identify each member of a particle set to which a specific capture probe has been immobilized. Another exemplary platform uses holographic barcodes to identify cylindrical glass particles. For example, Chandler et al. (U.S. Pat. No. 5,981,180) describes a particle-based system in which different particle types are encoded by mixtures of various proportions of two or more fluorescent dyes impregnated into polymer particles. Soini (U.S. Pat. No. 5,028,545) describes a particle-based multiplexed assay system that employs time-resolved fluorescence for particle identification. Fulwyler (U.S. Pat. No. 4,499,052) describes an exemplary method for using particle distinguished by color and/or size. U.S. Publication Nos. 2004-0179267, 2004-0132205, 2004-0130786, 2004-0130761, 2004-0126875, 2004-0125424, and 2004-0075907 describe exemplary particles encoded by holographic barcodes.

U.S. Pat. No. 6,916,661 describes polymeric microparticles that are associated with nanoparticles that have dyes that provide a code for the particles. The polymeric microparticles can have a diameter of less than one millimeter, e.g., a size ranging from about 0.1 to about 1,000 micrometers in diameter, e.g., 3-25 μm or about 6-12 μm. The nanoparticles can have, e.g., a diameter from about 1 nanometer (nm) to about 100,000 nm in diameter, e.g., about 10-1,000 nm or 200-500 nm.

An intact substrate can be covalently or non-covalently bound to a support. A variety of chemical reactions useful for covalently attaching a substrate to a support are well known to those skilled in the art (see, for example, Hartmann et al. (2002) J. Mater. Res. 17(2):473-478). Illustrative examples of functional groups useful for covalent attachment of substrates to a support include alkyl, Si—OH, carboxy, carbonyl, hydroxyl, amide, amine, amino, ether, ester, epoxides, cyanate, isocyanate, thiocyanate, sulfhydryl, disulfide, oxide, diazo, iodine, sulfonic or similar groups having chemical or potential chemical reactivity. Illustrative examples of binding partners useful for non-covalent attachment of substrates to a support include antibodies, antibody-like materials, and agents, e.g., that are capable of binding to antibodies such as, but not limited to, staphylococcal protein A or protein G.

In many of the methods described herein, a support-bound intact substrate is distinguishable from any support-bound product, such as a support-bound product portion. As such, a support-bound intact substrate can be detected without substantially detecting a support-bound product.

In some embodiments, an intact substrate can be detected by its interaction with a detectable tag. The detectable tag can interact directly or indirectly with the intact substrate. Examples of direct interaction with detectable tags include binding of the intact substrate to a fluorescently labeled intact-substrate-binding partner, such as an antibody, antibody-like materials, protein, nucleic acid, small molecules (such as an aptamer), and the like. As used herein, the term “intact-substrate-binding partner” means a molecule or complex that binds selectively to an intact substrate (e.g., without substantially binding to prevalent molecules unrelated to the intact substrate). The intact-substrate-binding partner generally will not bind substantially to a support-bound product portion, However, for many applications, the intact-substrate binding partner can be capable of binding to a free product portion, so long as the free product portion is separable from the support since in these cases the intact-substrate-binding partner is used to detect the intact substrate on the support. In a sample prepared for a detection step, the binding partner is principally associated with intact substrate.

Examples of indirect interaction with detectable tags include binding of the intact substrate to a primary intact-substrate-binding partner, which in turn, interacts with a secondary reagent that has a detectable tag and binds to the intact-substrate-binding partner.

A detectable tag can have an intrinsic detectable signal, or can be capable of generating a detectable signal. As such, a detectable tag can contain a detectable moiety, such as a luminescent moiety, fluorescent moiety, radioactive moiety and the like. Examples of fluorescent moieties include phycoerythrin A, fluorescein, rhodamine, 7-nitro-2,1,3-benzoxadiazol-4-yl, N-methylanthranyl and 2-cyanonaphtho[2,3-c]-2H-pyrrolyl, a transition metal chelate, such as a europium chelate, for example, a europium chelate of 1-(p-aminobenzyl)diethylenetriamine-N1,N1,N2,N3,N3-pentaacetic acid, and the like. Examples of radioactive moieties include ³²P, ³³P, ³⁵S, ¹²⁵I, or ³H.

In some embodiments where substrate modification is to be detected (see above), a support-bound product portion can be detected with a “support-bound product-binding partner.” Similarly, where a free product portion of a modified substrate is produced by an enzymatic activity and is to be detected, the free-product portion can be detected with a “free-product-binding partner.”

It is understood that suitable intact-substrate-binding partners, free product-binding partners, and support-bound-product-binding partners will vary for particular substrates and enzymatic activities. For example, a nucleic acid intact substrate can be bound by a intact-substrate-binding partner that is a nucleic acid sequence complementary to the intact substrate. Where the intact substrate is a polypeptide, the intact-substrate-binding partner can be an antibody specific for the polypeptide or an epitope contained therein. For example, a support-bound intact kinase substrate contacted with its cognate kinase, and thus phosphorylated, could be preferentially detected using, e.g., antibodies that bind to phosphorylated substrate (modified or support-bound product portion of the intact substrate) as compared to the non-phosphorylated (unmodified or intact) substrate. An exemplary intact-substrate-binding partner (streptavidin-phycoerythrin, which binds to intact biotinidase substrate), is described in Example 1.

A detectable tag also can be capable of generating a detectable signal, for example, upon addition of an activator, substrate, amplifying agent and the like. Well known detectable tags capable of generating a detectable signal include enzyme-labeled antibodies. Exemplary enzymes include horse radish peroxidase, beta-galactosidase, and beta-glucuronidase. As an example, an intact-substrate-binding-partner can be tagged with horse radish peroxidase. Upon formation of a target molecule-capture agent complex, detection can then be performed using any of a wide range of well known methods for detecting horseradish peroxidase, including 3,3′,5,5′-tetramethylbenzidine (TMB)-based chromogenic methods, 3,3′-diaminobenzidine tetrahydrochloride (DAB)-based chromogenic methods, 3-amino-9-ethylcarbazole (AEC)-based chromogenic methods, Amplex Red dye-based fluorogenic methods, enhanced luminol-based chemiluminescence reactions, electron paramagnetic resonance-based detection of free tyrosyl radical, luminescent semiconductor crystal (quantum dot) and tyramide signal amplification. Thus, an intact substrate can be detected by observing a physiochemical property as well as by observing a functional activity of the detectable tag. A physicochemical property such as mass, fluorescence absorption, emission, energy transfer, polarization, anisotropy, and the like, can also be observed, for example.

When an intact substrate or detectable tag contains a luminescent or dye component, detection can be by visual observation on a UV transilluminator, or by using a UV-based charged coupled device (CCD) camera detection system, a laser-based gel scanner, a xenon-arc-based CCD camera detection system, a Polaroid camera combined with a UV-transilluminator as well as a variety of other devices used for detecting luminescence.

When an intact substrate or detectable tag contains a radioactive component, detection can involve the use of a scintillation or liquid scintillation counter, gamma spectroscopy, Geiger counter, or certain types of X-ray photographic film (e.g., Kodak X-OMAT AR film, Kodak, Rochester, N.Y.).

A sample can be any composition. The content of the sample can be known or unknown. In many cases, a sample contains or is suspected of containing one or more enzymes. For example, a sample can be derived from an organism or man-made source of enzyme. A sample can be, for example, a specimen obtained from an individual or can be derived from such a specimen. For example, a sample can be a tissue section obtained by biopsy, or cells that are placed in or adapted to tissue culture. A sample can also be, or contain, a biological fluid specimen such as urine, blood, plasma, serum, saliva, semen, sputum, cerebral spinal fluid, tears, mucus, sweat, milk, semen, and the like. Biological samples can also be, or contain, fluid from ulcers or other surface eruptions such as blisters and abscesses or can be extracts of tissues from biopsies of normal, malignant, or suspect tissues. A sample can be further fractionated, if desired, to a fraction containing particular components or cell types. For example, a blood sample can be fractionated into serum or into fractions containing particular types of blood cells such as red blood cells or white blood cells (leukocytes). If desired, a sample can be a combination (pool) of samples from an individual such as a combination of a tissue and fluid sample, and the like.

A sample can be processed to facilitate detection of enzymes. For example, if the sample includes cells or other biological structures, the sample can be treated with freeze/thaw treatment, drying and rehydrating, a dounce, detergent or other methods. Releasing or solubilizing enzymes can also be used provided they do not interfere with the assay, e.g., by modifying the support-bound substrate.

For diagnostic purposes, the sample can be obtained from body fluids and tissues in which particular enzymes being tested are typically expressed. As non-limiting examples, a whole blood sample can be used when biotinidase is the enzyme; urine can be used when branched-chain ketoacid dehydrogenase is the enzyme; and a cell culture sample can be used when the enzyme is a recombinant enzyme. However, generally any sample can be tested for any enzymatic activity.

Samples can be treated with customary care to preserve enzymatic activity. Suitable methods for obtaining samples that preserve the activity or integrity of enzymes in the sample are well known to those skilled in the art. Such methods include the use of appropriate buffers and/or inhibitors, including nuclease, protease and phosphatase inhibitors that preserve or minimize changes in enzymes in the sample. Such inhibitors include, for example, chelators such as ethylenediamne tetraacetic acid (EDTA), ethylene glycol bis(P-aminoethyl ether) N,N,N1,N1-tetraacetic acid (EGTA), protease inhibitors such as phenylmethylsulfonyl fluoride (PMSF), aprotinin, leupeptin, antipain and the like, and phosphatase inhibitors such as phosphate, sodium fluoride, vanadate and the like. Inhibitors can be chosen such that they do not interfere with or only minimally adversely affect the enzymatic activity of interest. For example, if the enzymatic activity to be detected is a protease, methods for obtaining samples that preserve the activity or integrity of the enzyme would not include protease inhibitors that adversely affect the particular protease activity. Appropriate buffers and conditions for enzyme-containing samples are well known (see, for example, Ausubel et al. Current Protocols in Molecular Biology (Supplement 47), John Wiley & Sons, New York (1999); Harlow and Lane, Antibodies: A Laboratory Manual (Cold Spring Harbor Laboratory Press (1988); Harlow and Lane, Using Antibodies: A Laboratory Manual, Cold Spring Harbor Press (1999); Tietz Textbook of Clinical Chemistry, 3rd ed. Burtis and Ashwood, eds. W. B. Saunders, Philadelphia, (1999)).

A sample can be processed to eliminate or minimize the presence of interfering substances, as appropriate. If desired, a sample can be fractionated by a variety of methods well known to those skilled in the art, including subcellular fractionation, and chromatographic techniques such as ion exchange, hydrophobic and reverse phase, size exclusion, affinity, hydrophobic charge-induction chromatography, and the like (Ausubel et al. supra, 1999; Scopes, Protein Purification: Principles and Practice, third edition, Springer-Verlag, New York (1993); Burton and Harding, J. Chromatoqr. A 814:71-81 (1998)).

For use in a method described herein, a sample can be in a variety of physical states. For example, a sample can be a liquid or solid, can be dissolved or suspended in a liquid, can be in an emulsion or gel, and can be absorbed onto a material. As a non-limited example, a sample can be a liquid blood sample, liquid serum sample, liquid white blood cell sample, dried blood, serum, or white cell sample, or such a sample absorbed onto a paper or polymer substrate. Example 1 describes use of a dried blood sample.

The methods described herein are carried out under conditions that allow an enzyme to act on a substrate. Conditions under which proteins retain activity are well known to those skilled in the art and generally include roughly physiologically salt levels, a buffering agent, and a temperature in the range of 4-37° C. For a chosen enzyme, a sample can be adjusted or placed into a solution or environment to have a specified characteristic such as a specified pH, salt concentration, surfactant property, viscosity and the like. The ability of an enzyme to act on a substrate can be improved, enhanced and/or stabilized in the presence of sample ingredients such as inorganic salts, alcohols, detergents and surfactants, if desired.

In some embodiments, the methods described herein include separating a support-bound intact support from a sample and/or other components of a reaction mixture such as products, enzymes, free intact-substrate-binding-partners, detectable tags, sample and reaction buffer constituents and the like, thereby preparing the intact substrate for detection. Separation of the intact substrate can be achieved by a variety of well known means. When the intact substrate is attached to a support, a separation can be performed by removing a liquid phase from the support and/or by washing the support to remove a liquid phase, gel, colloidal, or other type of non-liquid phase from the support. The separation can occur in a variety of formats, such as column, membrane, particle separation by gravity, vacuum, magnetic or other force, and the like. A method described herein can be used in a diagnostic test relating to a metabolic disorder (e.g., a disorder in which one or more enzymatic activities are absent in a subject), if desired. Metabolic disorders can include, for example, acid lipase disease, amyloidosis, Barth Syndrome, biotinidase deficiency, carnitine palitoyl transferase deficiency type II (CPT-II), central pontine myelinolysis, Farber's disease, Fabry's disease, Pompe's disease, glucose-6-phosphate dehydrogenase deficiency, gangliosidoses, trimethylaminuria, Lesch-Nyhan Syndrome, methylmalonic aciduria (MMA), mucopolysaccharidosis, mucolipodosis, multiple CoA carboxylase deficiency (MCCD), nonketotic hyperglycinemia (NKH), propionic acidemia (PROP), type I glycogen storage disease, hyperoxaluria, oxalosis, 3-methylcrotonyl-COA carboxylase deficiency (3MCC), 3-OH 3-CH3 glutaric aciduria (HMG), arginosuccinic acidemia (ASA), beta-ketothiolase deficiency (BKT), 21-hydroxylase deficiency, CBS deficiency, maple syrup urine disease (MSUD), phenylketonuria (PKU), and very long-chain acyl-CoA dehydrogenase deficiency (VLCADD). Individuals of all ages can be affected by a metabolic disorder. Therefore, a sample used in a method described herein can be obtained from an individual of any age, including a neonate, a newborn child, a toddler, an adolescent, or an adult. For example, a newborn baby can be from 0 to 4 months old (e.g., from 0 to 5 days old, from 0 to a week old, from 0 to 2 weeks old, from 0 to 4 weeks old, from 0 to 6 weeks old, from 0 to 2 months old, from 0 to 10 weeks old, from 0 to 3 months old, or from 0 to 4 months old).

Symptoms of metabolic disorders are varied and are generally specific to a particular metabolic disorder. For example, symptoms of biotinidase deficiency (BIOT) include hypotonia, ataxia, seizures, developmental delay, alopecia, seborrheic dermatitis, hearing loss, optic nerve atrophy, and metabolic acidosis. Thus, a subject (e.g., a human newborn baby) suspected of having a biotinidase deficiency can be a subject who presents one or more of the aforementioned symptoms of biotinidase deficiency. A subject suspected of having a biotinidase deficiency can be a subject with a genetic predisposition for biotinidase deficiency (e.g., a family history of BIOT or a known mutation in the biotinidase gene).

The methods described herein are applicable to human and non-human individuals. As such, the methods can be used on a sample obtained, for example, from any veterinary or research subject for which a metabolically relevant enzyme and is known. Exemplary non-human animals include a horse, dog, cat, rabbit, rat, mouse, fish, turtle and lizard.

The methods of the invention also are applicable to lower organisms, such as yeast, archebacteria and bacteria, and plants, so long as a sample containing an enzyme can be obtained. In some instances, the presence of a microbial enzyme (e.g., from a pathogenic microorganism) can be assessed in a sample obtained from a mammal (e.g., a human), e.g., to test for an infection. For example, infection of a human subject by human immunodeficiency virus (HIV) could be assessed by detecting the activity of one or more HIV-specific enzymatic activities (e.g., HIV protease or HIV integrase) in blood from an individual. Infection of a subject by a bacterium (e.g., salmonella, streptococcus, or staphylococcus) or fungus (e.g., yeasts, tinea pedis, tinea cruris, or tinea capitis), similarly, could be determined by detecting the presence of one or more microbial enzymes present in a sample (e.g., blood, skin, or stool samples) obtained from the subject.

The methods described herein can also be used a diagnostics for other conditions such as hepatic or myocardial injury. For example, the presence or elevated amount of one or more lysosomal enzymes (e.g., acid and neutral protease, N-acetylglucosaminidase, or acid phosphatase) in the blood of a subject (e.g., a human patient) can be assessed in order to determine if the subject has hepatic injury such as hepatic cirrhosis or necrosis. The presence or elevated amount of angiotensin-converting enzyme (ACE) in blood, as determined from the methods described herein, can be used to detect injury to the heart muscle (e.g., ischemic injury due to or following a myocardial infarction).

The methods described herein can also be used in screening assays to identify compounds that modulate (e.g., inhibit or enhance) the activity of a given enzymatic activity. For example, the methods can be performed using samples containing a known amount of an enzymatic activity (enzyme), capable of modifying a cognate intact substrate, without and with a candidate compound. In one embodiment, a reduced amount of detectable enzymatic activity in the presence of the candidate compound as compared to the absence of the candidate compound in a sample, indicates the candidate compound is a compound that inhibits the enzymatic activity acting on the intact substrate. In other embodiments, an increased amount of detectable enzymatic activity in the presence of the candidate compound as compared to in the absence of the candidate compound indicates that the candidate compound is a compound that enhances the enzymatic activity acting on the intact substrate.

The methods described herein can be performed in a multiplexed format such that a plurality of samples and/or a plurality of enzymatic substrates are assayed simultaneously. An illustrative multiplexed format involves using physically and/or chemically coded particles. Use of coded particles in multiplexed formats has been described, for example, in U.S. Pat. No. 6,649,414 and U.S. Pat. No. 6,939,720. Because the codes allow particles to be distinguished from each other, a plurality of distinct particles can be present in a single reaction mixture, allowing a plurality of different samples or different enzymes to be assayed simultaneously. Codes on particles can correspond, for example, to sample origins, particular enzymes to be assayed, particular substrates present, and the like, depending on the experimental goal of the user (see above).

In some embodiments described herein, for example where magnetic particles are used, exemplary methods for performing such assays (transferring magnetic particles from well to well of a multi-step reaction) are set forth below.

Packages and kits that include one or more compositions for carrying out the described methods are also provided. A package can contain one or more of: an intact substrate, a support, a support-bound intact substrate, and/or reagents useful for forming a support-bound intact substrate. A package can include two or more support-bound intact substrates, if desired. A package can contain a variety of components in addition to a support-bound intact substrate. A package can contain, for example, a set of instructions for performing an assay, as well as guidance regarding temperature, buffer conditions and incubation time periods. A package can contain other components, such as one or more sample receptacles, such as microplates, sample fractionation devices, detectable tags, intact-substrate-binding partners, binding buffers, wash buffers, and the like. Those skilled in the art will be able to select suitable components for inclusion in a commercial package based on such exemplary factors as design of the assay protocol, the specific intact substrate to be detected, method of measurement to be employed once the assay has been performed, consumer price point, shipping and handling suitability and the like. The kits described herein can be used, for example, in the diagnosis of metabolic disorders such as, but not limited to, biotinidase deficiency or any other disorders described herein.

Also featured are methods of performing a multi-step assay (e.g., a multi-step assay for the detection of one or more enzymatic activities in a sample) within a single assay vessel using magnetic particles. The methods use a manipulator to transfer particles from one set of compartments to another. The transfers can be performed serially and can be combined with other steps, e.g., washing, agitation, binding reactions, and so forth. By transferring particles, the need to manipulate liquids is reduced.

For example, a plurality of reagents required to perform an assay are placed in adjacent wells of a multi-well plate. A manipulator, such as a magnetic probe described above, can then be used to transfer a set of particles (e.g., paramagnetic particles) from one well to another, generally with particle transfer to and from at least four wells being required to perform a complete assay. The magnetic particles, for example, can comprise a capture moiety as described above. For example, the particles are contacted with a first well to indirectly label a capture moiety, the a second well for a first wash step, then to a third well containing a fluorescent reporter, and finally to a fourth well as a second wash step.

This approach also allows all of the components of a multi-step assay to be provided within the wells of a microplate. For example, kits can be produced with different sets of solutions in different respective columns of a microplate. Users of such kits can transfer particles from one column to another. In certain embodiments, the first column can include the magnetic particles (either a homogenous set, or a set of encoded magnetic particles wherein different capture moieties are associated with a code). When the kit is used, different samples can be placed into the first compartment of each row, i.e., in the compartments containing the magnetic particles. After binding or other reaction, the particles can be transferred to the second column, e.g., containing a washing reagent. In some embodiments, one of the columns includes a detection reagent (a detection reagent comprising a detectable tag), or a mixture of different detection reagents or other reagents for performing and/or monitoring enzyme reactions (e.g., those useful for detection; see above). After further washing particles can be evaluated, e.g., by using a flow cytometers to evaluate multiple different detectable tags or detectable enzymatic activities.

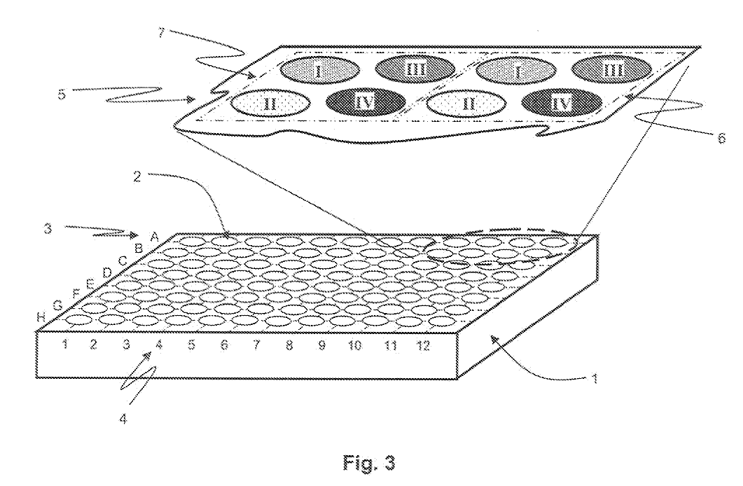

As a further illustration, FIG. 3 depicts an exemplary assay microplate. As used herein a “microplate” is a multi-well assay vessel made according to standards set by the Society of Biomolecular Screening, which typically has 96 or 384 wells (96 wells as shown in FIG. 3). The wells of the microplate are laid out in rows and columns, wherein the rows are designated with letters and the columns with numbers (see FIG. 3, numerical indicators 3 and 4 respectively). The figure includes a magnified view of its wells divided into subsets, wherein the wells of each subset contain a collection pre-loaded reagents loaded into the wells according to a pattern (see numerical indicator 6). Two of the subsets (numerical indicators 6 and 7) are shown in the magnified view; which subsets each contain four wells, and each well in the subset is numbered in the figure with Roman numeral I through IV. According to one embodiment of the present invention, each subset of wells would contain all of the reagents required to run a multi-step non-homogeneous assay.

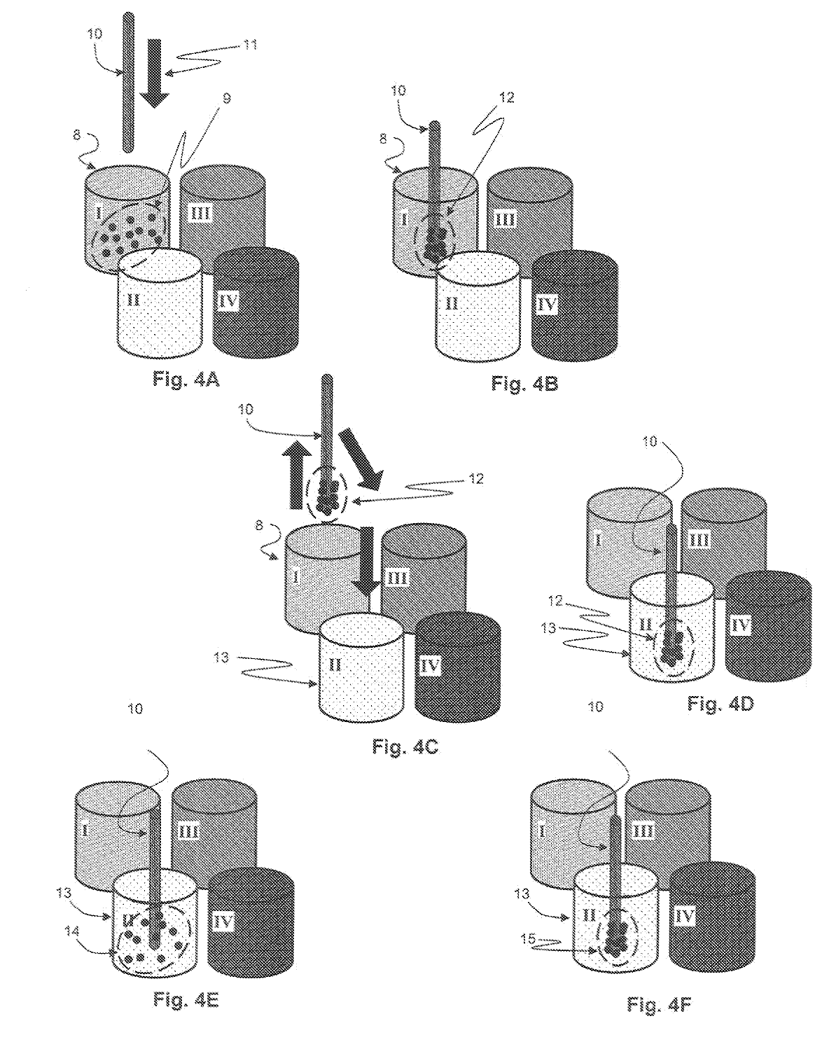

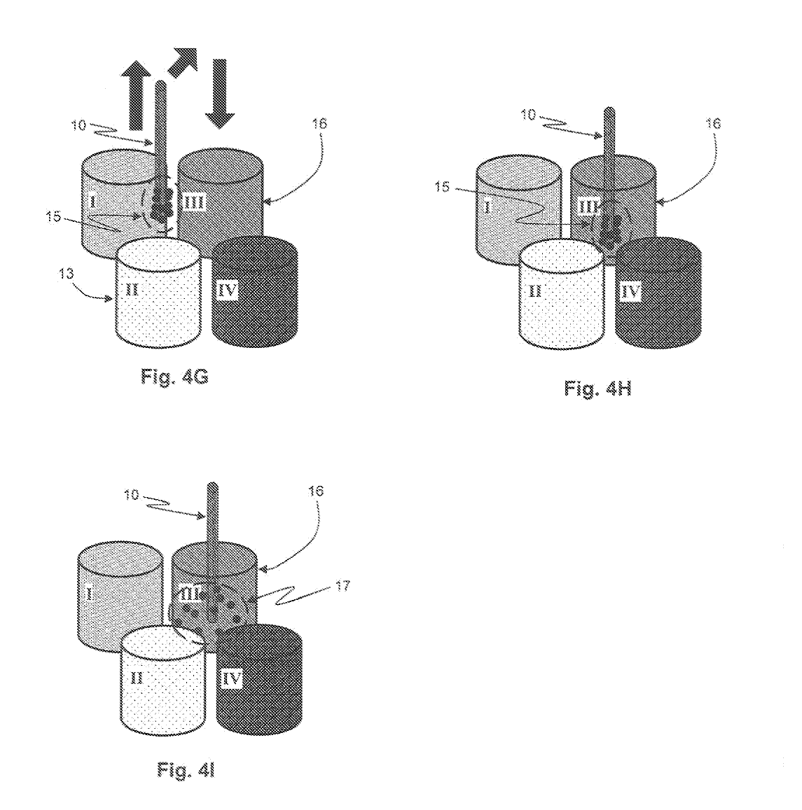

FIGS. 4A-4I depict an exemplary set of particle transfer steps according to one aspect of the present invention. First, a sample is incubated with a magnetic particle set comprising capture moiety in the I well (FIG. 4A). For example, the magnetic particle set can contain paramagnetic polymer particles for use in the Luminex xMAP multiplex assay system. A magnetic particle manipulator probe is then inserted into the I well (as shown in FIG. 4B). Next, the magnetic probe is activated, drawing the particle set into contact with the probe for subsequent transport (see FIG. 4C, numerical indicators 10 and 12). Motion of the probe in the plane of the microplate to sweep it through much of the volume of the well speeds up the collection of the particles by briefly bringing the probe very close to each particle, dramatically increasing the magnetic attraction. The probe carrying the particle set is then raised out of the I well, translated to a position above the II well (see FIG. 4C), and then lowered into the II well (see FIG. 4D). Next, the probe is deactivated and the particles are released from the probe, allowing them to disperse in the liquid medium of the II well (see FIG. 4E). This dispersion and suspension of the particles allows the capture moieties immobilized upon the particle surfaces to react with the assay media with kinetics approaching those of the given solutions. Following this reaction step, the magnet of probe is re-actuated and used to collect the particles at the probe's surface.

The particles are then transferred from the II well to the III well (as described above), where they are again dispersed and suspended. This operation can continue through all of the wells in each subset until the reaction is completed.

An example of an assay that can be performed using the multi-step assay methods described above is a sandwich immunoassay (also see above). For example, in an immunoassay with four steps in corresponding wells I-IV, the I well would contain a capture antibody (a first detection reagent) in buffer. Well II would contain a enzyme-labeled (detectably labeled) secondary antibody (a second detection reagent). Well III would contain a suitable detection solution, and well IV would contain a wash buffer from which assay particles could be, optionally removed and, analyzed. In this example, the 96-well microplate would have the capacity for 24, 4-step sandwich assays.

The magnetic particles, enzymatic substrates (e.g., a biotinidase substrate), exemplary assays, magnetic probes, and detection reagents can be any of those described herein.

It is understood that modifications that do not substantially affect the activity of the various embodiments of this invention are also included within the definition of the invention provided herein. Accordingly, the following examples are intended to illustrate but not limit the present invention.

### EXAMPLE 1

### Detection of Biotinidase Activity in Blood Samples

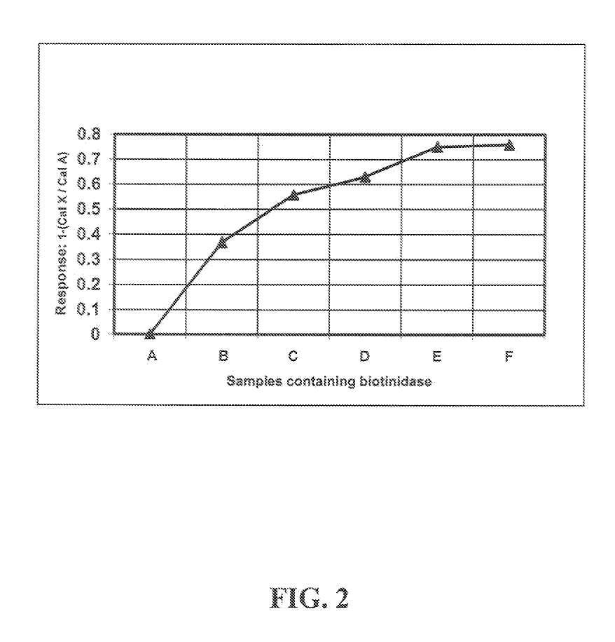

To detect the presence of biotinidase activity in blood samples, five 3.2 mm blood spot disks containing increasing amounts of biotinidase (B through F) and negative control disk (A) were incubated overnight at 37° C. with phosphate buffered saline and Luminex®-type paramagnetic beads conjugated with lysine-linker modified biotin. Streptavidin-phycoerythrin was added and allowed to incubate for 20 minutes at room temperature. The beads were then collected into a pellet by application of a magnet, and liquid was removed. The beads were then resuspended in buffer, and bead fluorescence was measured using a Luminex xMAP™ system using sheath fluid to follow the beads cytometically through a reader. The results are shown in FIG. 2. Samples contained various ratios of rabbit blood (which contains no biotinidase) and pig blood (which has biotinidase in amounts similar to normal humans). Sample A was pure rabbit blood; B was 20/80 pig/rabbit blood; C was 40/60 pig/rabbit blood; D was 60/40 pig/rabbit blood; E was 80/20 pig/rabbit blood; and F was 100% pig blood. The y axis corresponds to fluorescence signal from a Luminex xMAP 100 instrument normalized to the “Sample A” signal and subtracted from one (i.e., “1-Cal X/Cal A” in FIG. 1).

This example shows that biotinidase activity present in a blood sample can be assessed by detecting intact substrate present on a Luminex-type bead.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit, and scope of the present invention. All such modifications are intended to be within the scope of the present invention.

What is claimed is:

1. A method for evaluating multiple enzymatic activities in a sample, the method comprising:
providing a mixture of encoded particles comprising particles having an attached enzymatic substrate, the enzymatic substrate being identifiable by a particle code, wherein the mixture includes particles for a plurality of different enzymatic substrates;
contacting the mixture of particles with a sample; and
detecting modification of at least one of the enzymatic substrates attached to the particles for at least some of the encoded particles.

2. The method of claim 1, wherein modification of at least one of the enzymatic substrates attached to the particles produces a particle-bound product portion from the enzymatic substrate.

3. The method of claim 2, wherein the detecting comprises contacting the mixture of particles to a mixture comprising one or more enzymatic substrate-binding partners, wherein the one or more enzymatic substrate-binding partners are detectably-labeled and detect support-bound product portions from enzymatic substrates.

4. The method of claim 1, wherein the detecting comprises contacting the mixture of particles to a mixture comprising one or more enzymatic substrate-binding partners, wherein the one or more enzymatic substrate-binding partners are detectably-labeled and detect unmodified enzymatic substrates

5. The method of claim 4, wherein the detecting further comprises evaluating interaction of enzymatic substrate binding partners with the particles and the particle codes using a flow cytometer.

6. The method of claim 1, wherein the detecting comprises determining the amount of modification of at least one of the particle-bound enzymatic substrates.

7. The method of claim 1, wherein the detection comprises detecting the presence or amount of at least one unmodified particle-bound enzymatic substrate.

8. The method of claim 1, wherein the particle is magnetic.

9. The method of claim 1, wherein at least one enzymatic substrate comprises a linker moiety.

10. The method of claim 1, wherein the mixture of particles is contacted with more than one sample in parallel.

11. A method for detecting hydrolase activity, the method comprising:
providing a hydrolase substrate bound to a support;
contacting the support-bound hydrolase substrate with a sample; and
detecting modification of the support-bound hydrolase substrate.

12. The method of claim 11, wherein the detecting comprises determining the amount of modification of the support-bound hydrolase substrate.

13. The method of claim 11, wherein the detection comprises detecting the presence or amount of unmodified support-bound hydrolase substrate.

14. The method of claim 11, wherein the support is a magnetic particle.

15. The method of claim 11, wherein the support is an encoded particle.

16. The method of claim 11, wherein the support is a magnetic and encoded particle.

17. The method of claim 11, wherein the hydrolase is a biotinidase.

18. The method of claim 17, wherein the hydrolase substrate comprises biotin or biocytin.

19. The method of claim 11, wherein the detecting comprises contacting the support-bound hydrolase substrate with a detectably-labeled hydrolase substrate-binding partner.

20. The method of claim 19, wherein the detectably-labeled hydrolase substrate-binding partner is labeled with a fluorescent moiety.

21. The method of claim 20, wherein the detectably-labeled hydrolase substrate-binding partner comprises a member of a specific binding-pair.

22. The method of claim 21, wherein the member of the specific binding-pair is biotin or streptavidin.

23. The method of claim 11, wherein a support-bound hydrolase substrate is contacted with more than one sample in parallel.

24. A method for detecting biotinidase activity, the method comprising:
providing a biotinidase substrate bound to a support;
contacting the support-bound biotinidase substrate with a sample; and
detecting modification of the support-bound biotinidase substrate.

25. The method of claim 24, wherein the detecting comprises contacting the support-bound biotinidase substrate with a detectably-labeled biotinidase substrate-binding partner.

26. The method of claim 25, wherein the detectably-labeled biotinidase substrate binding partner is labeled with a fluorescent moiety.

27. The method of claim 25, wherein the detectably-labeled biotinidase substrate-binding partner comprises streptavidin.

28. The method of claim 25, wherein the detectably labeled biotinidase substrate-binding partner comprises avidin.

29. A kit for detecting biotinidase activity, the kit comprising:
(i) a support-bound biotinidase substrate; and
(ii) instructions for detecting biotinidase activity.

30. A diagnostic method for evaluating biotinidase deficiency in a subject, the method comprising:
providing a biotinidase substrate bound to a support;
providing a sample from a subject;
contacting the support-bound biotinidase substrate with the sample; and
detecting whether modification of the support-bound biotinidase substrate has occurred.

31. The method of claim 30, further comprising
providing a result indicating whether the subject has a biotinidase deficiency, wherein the result is a function of the extent of reduction in modification of the support-bound biotinidase substrate, relative to a reference of normal control.

32. The method of claim 30, wherein the subject is a human.

33. The method of claim 32, wherein the human is a newborn child.

34. The method of claim 30, wherein the subject is one having a genetic predisposition to a biotinidase deficiency.

35. The method of claim 30, further comprising
after evaluating the subject for a biotinidase deficiency, administering to the subject a therapeutically effective amount of biotin.

36. A method of detecting an enzymatic activity that catalyzes a reaction, the method comprising:
contacting an enzymatic substrate with a sample and a mixture comprising particles, wherein at least a portion of the particles preferentially bind to a reaction component; and
detecting the support-bound reaction component.

37. The method of claim 36, wherein reaction component is the enzymatic substrate.

38. The method of claim 36, wherein the reaction component is a reaction product produced from the enzyme-catalyzed reaction.

39. The method of claim 36, wherein the enzymatic substrate is contacted with the sample and then contacted with the mixture.

40. The method of claim 36, wherein at least some of the particles are encoded.

41. The method of claim 36, wherein at least some of the particles are magnetic.

42. The method of claim 36, wherein at least some of the particles are both magnetic and encoded.

43. The method of claim 36, wherein the enzymatic substrate and mixture are contacted to more than one sample in parallel.

44. The method of claim 36, wherein the enzymatic substrate comprises biotin or biocytin.

45. The method of claim 36, wherein the enzymatic substrate comprises a detectable tag.
